(19) United States
(12) Patent Application Publication
(10) Pub. No.: US 2024/0350270 A1
(43) Pub. Date: Oct. 24, 2024

(54) ARTICULATION COMPONENT OF A BONE JOINT IMPLANT

(71) Applicant: LOCI ORTHOPAEDICS LIMITED, Galway (IE)

(72) Inventors: Fiona MANGAN, Galway (IE); Gerry CLARKE, Galway (IE); Brendan BOLAND, Galway (IE)

(73) Assignee: LOCI ORTHOPAEDICS LIMITED, Galway (IE)

(21) Appl. No.: 18/688,247

(22) PCT Filed: Aug. 23, 2022
(86) PCT No.: PCT/EP2022/073523

§ 371 Date: Feb. 29, 2024

(30) Foreign Application Priority Data
Sep. 2, 2021 (EP) 21194631.4

(51) Int. Cl. A61F 2/30; A61B 17/72

(52) U.S. Cl. CPC A61F 2/30; A61B 17/72; A61F 2002/3065; A61F 2002/30654; A61F 2002/30662

(57) ABSTRACT

An articulation component (1, 100) is for a bone joint hemiarthroplasty implant having an intramedullary stem (50) with a socket (53). The articulation component has a proximal saddle (2, 110) for non-engaging abutment with a proximal bone and a ball (4, 104) for engaging in the stem socket (53). The saddle has a distally facing recessed surface (10, 110) for engagement with the stem proximally facing surface (55) to define limits of articulation, in one case a cone of motion of about 60° with minimal risk of socket dislocation. Also, the saddle has an enlarged lateral-most edge portion (11, 111, 211) on two diametrically opposed sides of the neck, for conformity with a patients' anatomy.

### INTRODUCTION

The present invention relates to bone joint implants.

Articulating hemi-arthroplasty implants are used to replace diseased joints in orthopaedic medicine. Examples include the carpometacarpal (“CMC”) joint, wrist, elbow, and shoulder.

One part of the implant is fixed into a bone such as the first metacarpal or radius. This intramedullary stem component may have at its proximal end an articular socket into which a head or “ball” is fitted. Examples are the implants described in WO2017/137607 and WO2020/193078.

The range of motion of the implant is determined by the interaction of the head and stem components.

In recent years, there have been an increasing number of implant designs that seek to maximise the mobility of implants. These include bipolar hips and thumb joint implants. The clinical logic is that as the range of motion is modelled against the patients' natural joint motions and anatomy, the better the clinical outcomes will be. Limited mobility can create edge loading and impingement, and so the implant must be designed for optimal head and stem interaction.

The present invention is directed towards providing for improved articulation to reduce the chances of an implant part contacting another part such as an intramedullary stem in a biomechanically inappropriate manner, and also to reduce risk of separation of the parts due to leverage on one part about another.

### SUMMARY

We describe an articulation component for a bone joint hemiarthroplasty implant having an intramedullary stem, the articulation component comprising a proximal saddle for non-engaging abutment with a proximal bone and an articulation head for engaging with the stem, wherein the saddle has a distally-facing recessed surface, said recessed surface being configured for engagement with an intramedullary stem to define limits of articulation.

In some examples, the recessed surface is annular around the neck. In some examples, the saddle has an enlarged lateral-most edge portion on two diametrically opposed sides of the neck.

In some examples, the lateral-most edge portion and the recessed surface form a ramped or shoulder surface providing continuity between the distally facing surface of the recessed portion and the edge portion.

In some examples, each lateral portion has a thickness which is at least greater than the thickness of the recessed portion where is adjoins the recessed portion by 0.2 mm.

In some examples, the recessed portion distally facing surface has on either side of the neck separate centres of curvature which are spaced apart in at least some proximal-distal planes through the articulation component.

We also describe a bone joint hemiarthroplasty implant comprising an intramedullary stem with a socket and a proximally facing abutment surface, and an articulation component of any example described herein, wherein the saddle distally facing surface recessed portion is configured for engagement with the stem proximally facing abutment surface to define limits of articulation.

In some examples, the recessed portion and the stem are configured to provide a cone of motion in in excess of 50°.

In some examples, the recessed portion and the stem are configured to provide a cone of motion in in excess of 55°.

In some examples, the recessed portion and the stem are configured to provide a cone of motion of about 60°.

We also describe an articulation component for a bone joint hemiarthroplasty implant having an intramedullary stem, the articulation component comprising a proximal saddle for non-engaging abutment with a proximal bone and an articulation head for engaging with the stem, wherein the saddle has a distally facing recessed surface of a recessed portion, said recessed surface being configured for engagement with the stem to define limits of articulation.

Preferably, the recessed surface is annular around the neck. Preferably, the saddle has an enlarged lateral-most edge portion on two diametrically opposed sides of the neck.

Preferably, the lateral-most edge portion and the recessed portion form a ramped or shoulder surface providing continuity between the distally facing surface of the recessed portion and the edge portion.

Preferably, each lateral portion has a thickness which is at least greater than the thickness of the recessed portion where is adjoins the recessed portion by 0.2 mm.

Preferably, the recessed portion distally facing surface has on either side of the neck separate centres of curvature which are spaced apart in at least some proximal-distal planes through the articulation component.

We also describe a bone joint hemiarthroplasty implant comprising an intramedullary stem with a socket and a proximally facing abutment surface, and an articulation component of any example, wherein the saddle distally-facing surface recessed portion is configured for engagement with the stem proximally-facing abutment surface to define limits of articulation.

Preferably, the recessed portion and the stem are configured to provide a cone of motion in in excess of 50°, more preferably in in excess of 55°, and in some examples preferably about 60°

### DETAILED DESCRIPTION OF THE INVENTION

The invention will be more clearly understood from the following description of some embodiments thereof, given by way of example only with reference to the accompanying drawings in which:

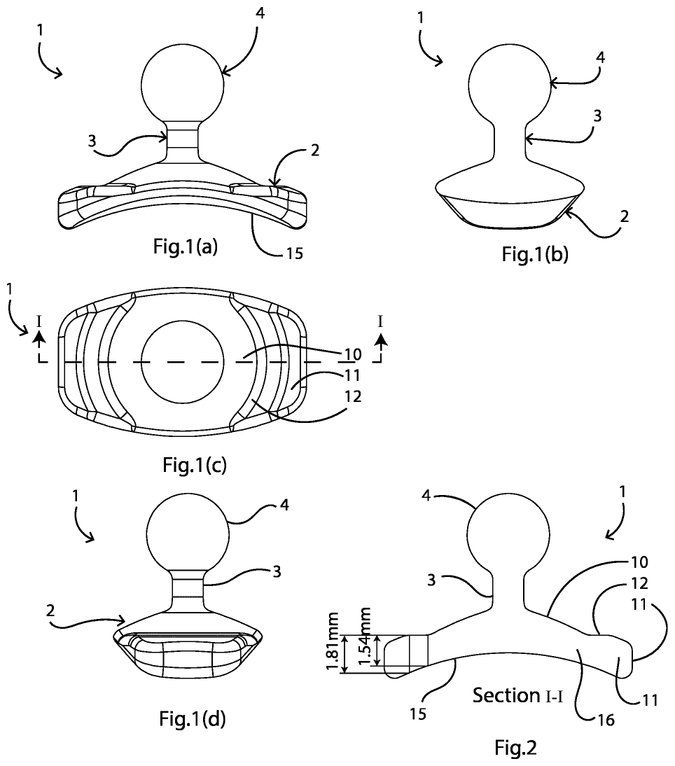

FIGS. 1(a) to (d) are a set of views of a bone joint implant articulation component of the invention, namely side, end sectional, top plan, and end views,

FIG. 2 is a cross-sectional view in the direction of the arrows I-I in FIG. 1(c),

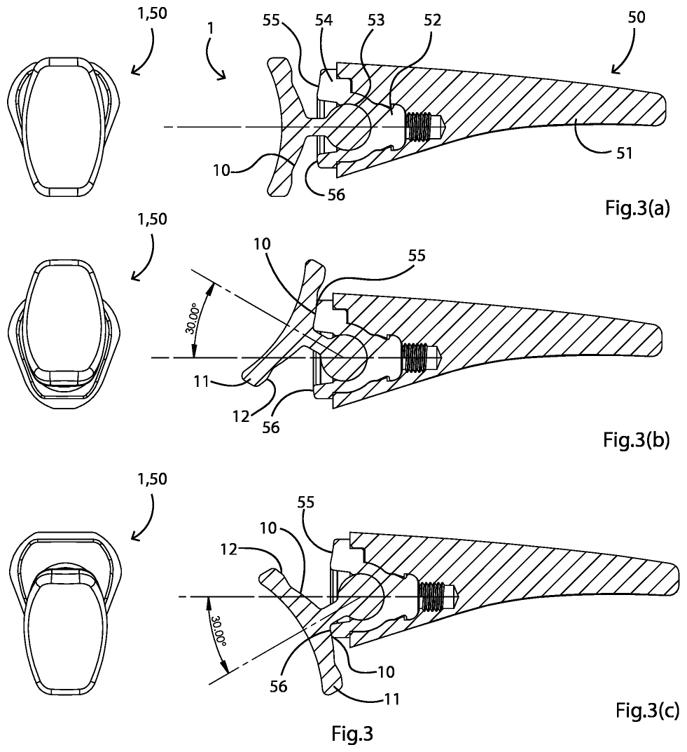

FIGS. 3(a), 3(b), and 3(c) are sectional views showing a hemiarthroplasty implant of the invention, incorporating the articulation component, in use,

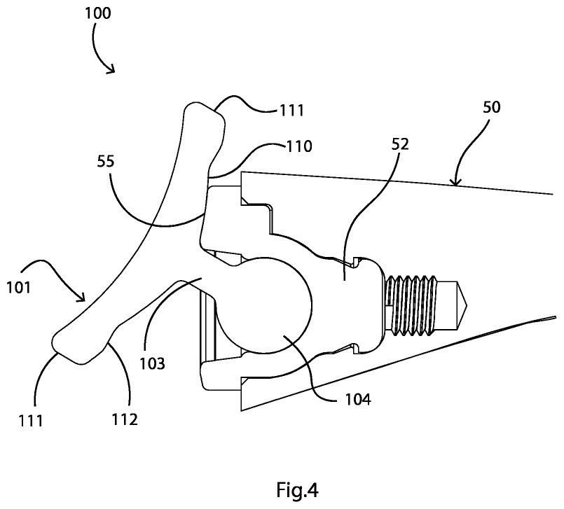

FIG. 4 is a diagrammatic side view showing an alternative hemi-arthroplasty joint articulation component of the invention;

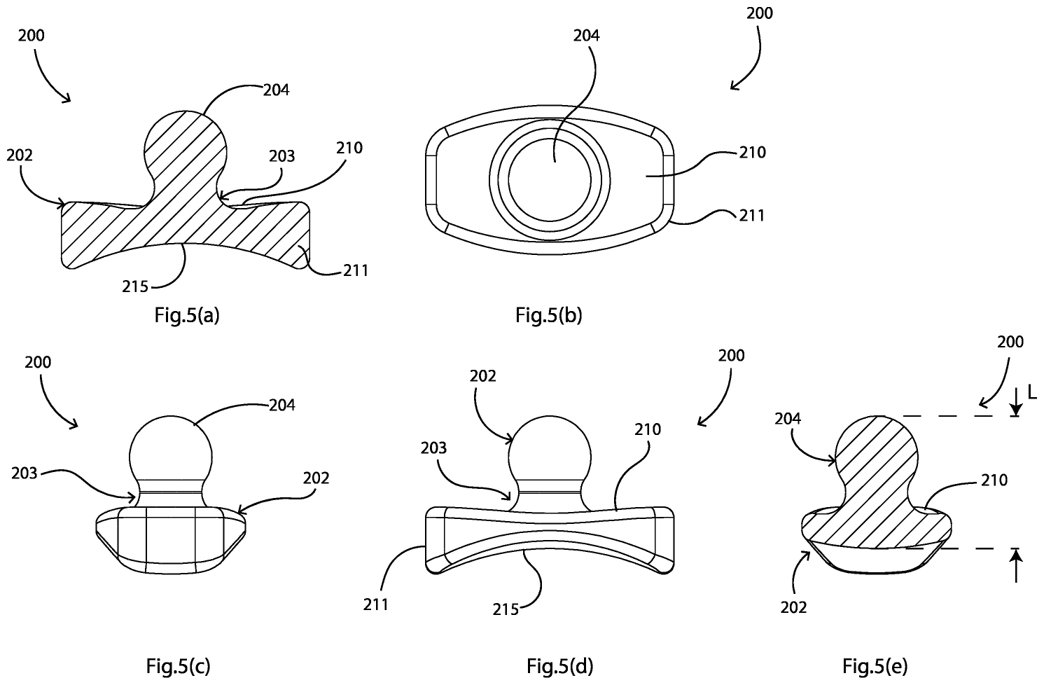

FIGS. 5(a) to 5(e) are drawings of an alternative articulation component in which:
FIG. 5(a) is a cross-sectional view through the component's widest dimension,
FIG. 5(b) is a plan view showing the distally facing surfaces,
FIG. 5(c) is a side view showing the component across its shortest lateral dimension,
FIG. 5(d) is a front view showing the component across its widest lateral dimension, and
FIG. 5(e) is a cross-sectional view through the component across the smallest lateral dimension; and

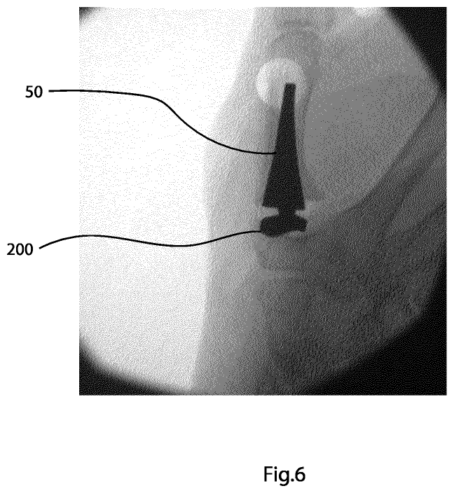

FIG. 6 is an X-ray image of an implant having a stem and an articulation component of FIG. 5 in place.

### TERMS

“Intramedullary engagement” means engagement within a medullary cavity formed or existing in the bone, where the cavity is generally but not exclusively formed along a longitudinal axis of the bone. In one embodiment, the intramedullary engagement fixture comprises a screw or nail or interference-fit stem, although other intramedullary fixtures are known. Typically, the screw is externally threaded. Intramedullary fixtures are sold by Smith & Nephew, Zimmer, Synthes and other suppliers. The engagement anchors the implant to the bone. In one embodiment, the medullary cavity is formed in a position that is offset towards a volar direction. The medullary cavity may be formed in a position offset from the anatomical and or biomechanical axis of the bone.

“Non-engaging abutment” means that the proximal part is not fixed to the first bone but is configured to abut the end of the bone in a manner that allows mutual sliding or translational movement. As an example, when the joint is a carpometacarpal joint in the thumb, the end of the trapezium bone has a twisted saddle shape (see FIG. 2 of Turker et al, Indian J Plast Surg. 2011, 44 (2): 308-316) and the platform is configured to rest upon this saddle and allow translational movement of the platform across the saddle. The proximal-facing surface of the curved saddle-shaped platform or “saddle” typically has a concave-convex shape, which has a concave curvature along a longitudinal aspect, and a convex curvature along a lateral aspect. This shape has been shown to provide an engagement that closely mimics the physiological situation and allows for natural flexion-extension articulation.

“Articulating coupling” means a coupling that allows articulation between the first and second parts of the implant. The specific type of coupling employed in the implant depends on the joint that is being treated with the implant, and in some cases the indication or severity of the indication. For example, when the implant is for treatment of an arthritic hinge joint, for example an elbow joint, the implant will generally comprise a hinge joint coupling. When the implant is for treatment of a saddle joint, for example a carpometacarpal joint, the implant will generally comprise a ball and socket joint or a universal joint. “Controlled articulation” means that the articulation is constrained to specific types of articulation.

### DESCRIPTION OF THE EMBODIMENTS

### Articulation Components Allowing Large Cone of Motion.

Referring to FIGS. 1(a) to (d) an articulation component 1 for a CMC joint hemiarthroplasty implant is illustrated. The component 1 comprises a proximal saddle 2 for sliding movement over a first bone, in this case the trapezium. The saddle 2 is joined by a neck 3 to a head 4, in this case a ball for insertion in a socket of an intramedullary stem, in this case for the metacarpal.

The function of the component 1 is akin in a general sense to that of an articulation component described in WO2020/193078 (Loci Orthopaedics Ltd), the contents of which are herein incorporated by reference.

The saddle 2 in this case has a proximally facing surface 15 (facing away from the ball 4) which has a combined convex and concave shape as described in WO2017/137607, and for the same purpose as described in that specification. Again, the contents of this document are herein incorporated by reference. Importantly, the saddle proximal surface 15 and the component 1 as a whole are shaped with a structure built to support the lateral pressure of an arch or span, for non-engaging abutment with the distally facing surface of a first bone, in this case the trapezium.

The saddle 2 has a recessed annular portion 16 with a distally facing annular recessed surface 10. On two diametrically opposed sides of the neck 3 there is an enlarged edge portion 11, having a distally facing surface linked with continuity to the recessed surface 10 by a tapered shoulder surface 12. The edge portions 11 are laterally of the recessed surface 10 on each lateral side as best illustrated in FIG. 1(c).

The specific dimensions of the saddle 2 and the recessed portion surface 10 are in, this particular example:
Radius of curvature of the recessed surface 10 as viewed in cross-section (FIG. 2), 14.1 mm. The centre of curvature of each of these surfaces as viewed in cross-section are 6.1 mm apart.
Saddle thickness as measured in the proximal-distal axis at the recessed portion 16, as shown in FIG. 2, 1.54 mm. This increases to 1.81 mm at the shoulder or ramped surface 12. Neck 3: 1.5 mm in diameter. Each lateral portion 11 has a thickness which is at least greater than the smallest thickness of the recessed portion 16 by at least 0.2 mm

In some examples, the proximal surface 15 of the saddle 2 ha a single radius of curvature and centre of curvature, and the surface 10 as viewed in cross-section has separate centres of curvature as illustrated.

As shown in FIGS. 3(a) to 3(c) an implant of the invention comprises the articulation component 1, and a metacarpal intramedullary stem 50 having a main stem body 51 with a proximal liner 52 having a socket 53 and an annular flange 54. The full stem component 50 is as described in WO2020/193078. However, in the invention there is considerable additional articulation of the saddle 2 about the stem 50 because the recessed surface 10 is located for abutment with the proximal-facing annular surface 55 of the stem 50. As shown most clearly in FIG. 3(b) the full extent of rotation in the plane of the page is 30° from a proximal-distal longitudinal axis through the implant. There is an equivalent extent of rotation in this plane on the opposed side, giving a total angle of articulation in this plane of 60°. There is also this same extent of rotation in the other planes, giving a whole cone of rotation range of motion (“ROM”) of about 60° around 360°.

This degree of articulation is achieved while still having the benefit of a desired volume/bulk of material to allow the implant to mimic the natural thumb joint. The regions of the saddle laterally closer to the neck and further from the neck are enlarged, with larger dimensions in the proximal-distal direction.

The recessed surface 10 may be regarded as a circular groove which matches the proximal-facing stem surface 55, in which the contact surfaces conform to each other.

The proximal surface of the saddle 2 is saddle-shaped with one surface convex while the other is concave, however, the distal surfaces have a general dome shape to conform with the stem proximally facing surface 55. This allows for more range of motion without the need to redesign the stem, thereby allowing the liner and the stem in general to have an optimum shape for fitting to the metacarpal and filling the optimal space in the hand of the patient.

Despite the large cone of motion there is little chance of dislocation, with a better distribution of contact forces over non congruent forces. Risk is reduced due to a greater range of motion before the head can get into a “lever out” position. This is because it increases the distance that the ball, and hence the head, can rotate within the joint. Also, it reduces the risk of the head 4 becoming trapped at an extreme end of motion position causing a build-up of counter reaction forces that may cause dislocation of the head 4 from the socket 53. Physiologically, it allows more movement of the metacarpal in relation to the ‘socket’ axis and point of rotation. These features are achieved without losing any of the benefits of movement over the trapezium.

Also, it is envisaged that there is a greater potential for mobility in the joint, potential use with a wider population, better suitability for natural subluxation conditions, while maintaining thickness in the saddle head and thereby maintaining strength.

The thickness of the saddle can also be modified to increase the distraction distance without affecting the head and stem articulation.

In another example shown in FIG. 4 an articulation component 100 has a proximal saddle 101, a ball 104, and a neck 103. In this case however the saddle 101 has a wider configuration, with lateral-most ends 111 being larger than those of the saddle 2. This drawing also shows the abutment of the recessed surface 112 with the flange in the stem for one extremity of motion.

### Articulation Components Confining Cone of Motion.

Referring to FIG. 5 an articulation component 200 is also for a CMC joint hemiarthroplasty implant. The component 1 comprises a proximal saddle 202 for sliding movement over a first bone, again in this case the trapezium. The saddle 202 is joined by a neck 203 to a head 204, in this case a ball for insertion in a socket of an intramedullary stem for the metacarpal.

As for the component 1 described above the function of the component 200 is akin in a general sense to that of an articulation component described in WO2020/193078 (Loci Orthopaedics Ltd).

The saddle 202 in this case has a proximally facing surface 215 (facing away from the ball 204) which has a combined convex and concave shape. The saddle proximal surface 215 and the component 200 are shaped with a structure built to support the lateral pressure of an arch or span, for non-engaging abutment with the distally facing surface of a first bone, in this case the trapezium.

The saddle 202 has a distally facing annular recessed surface 210. On two diametrically opposed sides of the neck 203 there is an enlarged edge portion 211, having a distally facing surface linked with continuity to the recessed surface 210. The edge portions 211 are laterally of the recessed surface 210 on each lateral side. In this case there is no distinct sloped shoulder surface akin to the surface 12 of the component 1, rather there is a gradual slope from the outermost lateral side towards the neck 203. The component 200 also differs from the component 1 in that the neck 203 is short, from the distal-most tip of the ball 203 to the surface 215 which is aligned with the axis of the neck 203. This dimension is denoted L in FIG. 5(e), and in this case is 6.4 mm, whereas the equivalent dimension in the component 1 is 7.1 mm. The shorter neck provides that the cone of motion is smaller, and in this case is only 10°. However, the recessed surface 210 performs the same function as the surface 11 as illustrated in FIG. 3, providing an abutment surface for contact between the articulation component and the stem distal surface (55) to set the cone of motion and to allow optimum achieved while still having the benefit of a desired volume/bulk of material to allow the implant to mimic the natural thumb joint.

This surface 310 provides less range of motion than that of the other examples, less than 10°. This would mimic a thumb joint with less range of motion, allowing a surgeon to have options of range of motions 0-60 degree, with only the head needing to be the changing component (stem and liner stay the same). The neck was shortened by 0.7 mm in height (distraction distance now 4.4) and enlarged to a 3 mm thick neck to allow for this minimum range of motion. All examples of the invention have in common that there are at least two diametrically opposed enlarged edge positions, with larger dimensions in the proximal-distal direction than those regions closer to the neck. There is also a recessed surface for abutment with the intramedullary stem, this surface location in relation to the distance L providing a setting of the cone of motion. The arrangement of these enlarged lateral edge portions is particularly effective for a saddle with combined convex and concave proximally facing surfaces for translational movement over a bone such as the trapezium.

FIG. 6 shows an X-ray image of the articulation component 200 in place, articulating with a stem 50, with its ball 204 engaged in the socket 53 of the stem.

Components of embodiments can be employed in other embodiments in a manner as would be understood by a person of ordinary skill in the art. The invention is not limited to the embodiments described but may be varied in construction and detail. For example, the features of the liner May be integral with the stem distal part.

1. An articulation component for a bone joint hemiarthroplasty implant having an intramedullary stem, the articulation component comprising a proximal saddle for non-engaging abutment with a proximal bone and an articulation head for engaging with the stem, wherein the saddle has a recessed portion with a distally facing recessed surface, said recessed surface being configured for engagement with an intramedullary stem to define limits of articulation.

2. The articulation component of claim 1, further comprising a neck, wherein the recessed surface is annular around the neck.

3. The articulation component of claim 1, further comprising a neck, wherein the saddle has an enlarged lateral-most edge portion on two diametrically opposed sides of a neck.

4. The articulation component of claim 3, wherein the lateral-most edge portion and the recessed surface form a ramped or shoulder surface providing continuity between the distally facing surface of the recessed portion and the edge portion.

5. The articulation component of claim 3, wherein each lateral portion has a thickness which is at least greater than the thickness of the recessed portion where each lateral portion adjoins the recessed portion by 0.2 mm.

6. The articulation component of claim 1, further comprising a neck, wherein the recessed surface has on either side of the neck separate centres of curvature which are spaced apart in at least some proximal-distal planes through the articulation component.

7. A bone joint hemiarthroplasty implant comprising an intramedullary stem with a socket and a proximally facing abutment surface, and an articulation component comprising a proximal saddle for non-engaging abutment with a proximal bone and an articulation head for engaging with the stem, wherein the saddle has a recessed portion with a distally facing recessed surface, said recessed surface being configured for engagement with an intramedullary stem to define limits of articulation, and wherein the saddle distally facing recessed surface of the recessed portion is configured for engagement with the stem proximally facing abutment surface to define limits of articulation.

8. The implant of claim 7, in which wherein the recessed portion and the stem are configured to provide a cone of motion in in excess of 50°.

9. The implant of claim 7, wherein the recessed portion and the stem are configured to provide a cone of motion in in excess of 55°.

10. The implant of claim 8, wherein the recessed portion and the stem are configured to provide a cone of motion of about 60°.

11. The implant of claim 7, further comprising a neck, wherein the recessed surface is annular around a neck.

12. The implant of claim 7, further comprising a neck, wherein the saddle has an enlarged lateral-most edge portion on two diametrically opposed sides of a neck.

13. The implant of claim 12, wherein the lateral-most edge portion and the recessed surface form a ramped or shoulder surface providing continuity between the distally facing surface of the recessed portion and the edge portion.

14. The implant of claim 12, wherein each lateral portion has a thickness which is at least greater than the thickness of the recessed portion where it adjoins the recessed portion by 0.2 mm.

15. The implant of claim 7, further comprising a neck, wherein the recessed has on either side of a neck separate centres of curvature which are spaced apart in at least some proximal-distal planes through the articulation component.

16. The implant of claim 1, wherein the in which the recessed portion and the stem are configured to provide a cone of motion in in excess of 50°.

17. The implant of claim 1, in which the recessed portion and the stem are configured to provide a cone of motion in in excess of 55°.

18. The implant of claim 16, in which the recessed portion and the stem are configured to provide a cone of motion of about 60°.
